(19) United States
(12) Patent Application Publication
(10) Pub. No.: US 2025/0302947 A1
(43) Pub. Date: Oct. 2, 2025

(54) M-cell GP2-mediated lymphatic-targeted drug carriers

(71) Applicant: Hong Kong Baptist University, Hong Kong (CN)

(72) Inventors: Quanbin HAN, Hong Kong (CN); Quanwei ZHANG, Hong Kong (CN); Lifeng LI, Hong Kong (CN)

(21) Appl. No.: 18/865,746

(22) PCT Filed: May 25, 2023
(86) PCT No.: PCT/CN2023/096272

§ 371 Date: Nov. 14, 2024

Related U.S. Application Data
(60) Provisional application No. 63/346,903, filed on May 29, 2022.

(51) Int. Cl. A61K 39/39; A61K 9/00; A61K 39/00; A61K 47/61; A61K 47/68

(52) U.S. Cl. CPC A61K 39/39; A61K 9/0053; A61K 47/61; A61K 47/6807; A61K 2039/542; A61K 2039/55583; A61K 2039/572; A61K 2039/585

(57) ABSTRACT

The present invention provides a delivery agent including Radix Astragali polysaccharide (RAP) which modulates the immune system quickly and induces anti-cancer immune responses after oral administration or aerosol administration. Fluorescently labeled RAP is shown to be efficiently delivered to mucosa of small intestine or lung by transcytosis through microfold (M) cells and directly got into contact with follicle dendritic cells (FDCs) or lung dendritic cells. In addition, it is demonstrated that glycoprotein 2 (GP2)-deficient M cells fail to transport RAP and induce immune responses, suggesting GP2 of M cells is a specific transcytosis receptor of RAP. The present invention also provides that immunomodulatory polysaccharides could be directly transported into the mucosal immune system by M cells in a GP2-dependent way. These lymphatic-targeted macromolecules are potential delivery agent of poorly bioavailable small molecules, drugs and vaccines, or alike, via oral or aerosol drug administration.

### CROSS-REFERENCE TO RELATED APPLICATION

This application claims priority from a U.S. provisional patent application No. 63/346,903 filed on May 29, 2022, the disclosure of which is incorporated herein by reference.

### FIELD OF THE INVENTION

The present invention relates to traditional Chinese Medicine derived macromolecules, which trigger antitumor immunity via receptor-mediated lymphatic absorption route. In particular, the present invention provides a lymphatic-targeted agent to deliver poorly bioavailable drugs and vaccines via oral dosing or aerosol drug administration.

### BACKGROUND OF THE INVENTION

The gut wall barrier to macromolecules remains an unsolved challenge for developing orally-delivered macromolecular therapeutics. Recently proposed solutions have focused on highly engineered systems such as self-orienting microinjectors, but such complexity creates challenges for manufacture and regulation. The present inventors sought inspiration from polysaccharides derived from traditional herbal medicines that positively affect the immune system after oral dosing. These molecules present a pharmacology paradox in that they achieve systematic action in the human body despite very low oral bioavailability. Understanding how medicinal polysaccharides access the immune system could open new doors in the oral delivery of macromolecules and the modernization of TCM decoctions.

One potential route by which macromolecules could access the mucosal immune system is via transcytosis through microfold (M) cells. These specialized epithelial cells, located on the small intestine's immune sensor-Peyer's patches (PPs), use transcytosis to deliver intact pathogens and food molecules from the gut lumen to underlying dendritic cells to control immunity and tolerance. M cells express receptors on their luminal surface, which can potentially be targeted for drug delivery. Targeted transcytosis through M cells is a promising route for oral vaccination, especially bioactive macromolecules. Now that the small intestine is the main location where polysaccharides accumulate after oral administration, whether they could be transported across epithelial cell layer by M cells and how are warranted to figure out.

Basing on a delivery route targeting this M-cell receptor-mediated pathway to develop a delivery agent for a drug with poor bioavailability is an unmet need in the fields of pharmaceuticals and medicinal chemistry.

### SUMMARY OF THE INVENTION

Accordingly, it is an objective of the present invention is to provide traditional Chinese Medicine derived macromolecule triggers antitumor immunity. In particular, the present invention provides a targeting agent to deliver macromolecules drugs and vaccines via oral dosing or aerosol administration.

In a first aspect, the present invention provides a use of a Radix Astragali polysaccharide for increasing a bioavailability of a drug in a subject.

In certain embodiments, the Radix Astragali polysaccharide is a branched polysaccharide with an average molecular weight of 100-1500 kDa comprising Rha, Ara, Glc, Gal and GalA in a molar ratio of 0.01-0.03:1.00:0.20-10.00:0.01-0.50:0.01-0.30.

In certain embodiments, the branched polysaccharide has an average molecular weight of 1334 kDa consisting of Rha, Ara, Glc, Gal and GalA in a molar ratio of 0.03:1.00:0.27:0.36:0.30.

In certain embodiments, the branched polysaccharide comprises a backbone and one or more sidechains, wherein the backbone comprises 1,2,4-linked Rhap, α-1,4-linked Glcp, α-1,4-linked GalAp6Me, or β-1,3,6-linked Galp; and the one more sidechains comprise α-T-Araf, α-1,5-linked Araf with O-3, T-linked Araf, T-linked Glcp, and T-linked Galp.

In certain embodiments, the backbone of the branched polysaccharide consists of 1,2,4-linked Rhap, α-1,4-linked Glcp, α-1,4-linked GalAp6Me, β-1,3,6-linked Galp, with branches at O-4 of the 1,2,4-linked Rhap and O-3 or O-4 of β-1,3,6-linked Galp, and the sidechains are mainly α-T-Araf and α-1,5-linked Araf with O-3 as branching points, having trace Glc and Gal; terminal residues thereof are T-linked Araf, T-linked Glcp and T-linked.

In certain embodiments, the Radix Astragali polysaccharide is covalently bonded to the drug via an optional linker to form a covalent conjugate of the Radix Astragali polysaccharide and the drug.

In certain embodiments, the drug comprises a small molecule, a peptide, an antibody, an antibody fragment, or a vaccine.

In certain embodiments, the subject is a human or a non-human mammal.

In certain embodiments, the Radix Astragali polysaccharide is formulated into an oral administrable composition or an aerosolized composition.

In certain embodiments, the conjugate of the Radix Astragali polysaccharide and the drug is formulated into an orally administrable composition or an aerosolized composition.

In certain embodiments, the admixture is formulated into an orally administrable composition or an aerosolized composition.

In certain embodiments, the orally administrable composition is formulated into a solid, a powder, a liquid, a gel, a capsule, a tablet, a pill, a pellet, or a particle.

In certain embodiments, the aerosolized composition is formulated into particles or aerosols.

In certain embodiments, the drug is transported via a lymphatic system at small intestine mucosa or lung mucosa of the subject by an M-cell GP2-mediated transcytosis.

A second aspect of the present invention provides a delivery agent comprising a polysaccharide specifically targeting a receptor of microfold (M) cells, which when the polysaccharide is covalently bonded to a drug via an optional linker to form a polysaccharide drug conjugate, the resulting polysaccharide drug conjugate is capable of being transported via the lymphatic system of a subject via receptor-mediated transcytosis.

In certain embodiments, the polysaccharide comprises one or more Radix Astragali polysaccharides, wherein the one or more Radix Astragali polysaccharides are branched polysaccharides with an average molecular weight of 100-1500 kDa comprising Rha, Ara, Glc, Gal and GalA in a molar ratio of 0.01-0.03:1.00:0.20-10.00:0.01-0.50:0.01-0.30, wherein each of the branched polysaccharides comprises a backbone and one or more sidechains, wherein the backbone comprises 1,2,4-linked Rhap, α-1,4-linked Glcp, α-1,4-linked GalAp6Me, or β-1,3,6-linked Galp; and the one more sidechains comprise α-T-Araf, α-1,5-linked Araf with O-3, T-linked Araf, T-linked Glcp, and T-linked Galp.

Optionally, the polysaccharide can be admixed with the drug to form an admixture, and the resulting admixture is also capable of being transported via the lymphatic system of a subject via receptor-mediated transcytosis.

In certain embodiments, the branched polysaccharide has an average molecular weight of 1334 kDa consisting of Rha, Ara, Glc, Gal and GalA in a molar ratio of 0.03:1.00:0.27:0.36:0.30.

In certain embodiments, the backbone of the branched polysaccharide consists of 1,2,4-linked Rhap, α-1,4-linked Glcp, α-1,4-linked GalAp6Me, β-1,3,6-linked Galp, with branches at O-4 of the 1,2,4-linked Rhap and O-3 or O-4 of β-1,3,6-linked Galp, and the sidechains are mainly α-T-Araf and α-1,5-linked Araf with O-3 as branching points, having trace Glc and Gal; terminal residues thereof are T-linked Araf, T-linked Glcp and T-linked.

In certain embodiments, the M cells express glycoprotein 2 (GP2).

In certain embodiments, the drug is transported via the lymphatic system at small intestine mucosa or lung mucosa.

In certain embodiments, the drug comprises a small molecule, a peptide, an antibody, an antibody fragment, or a vaccine.

In certain embodiments, the subject is a human or a non-human mammal.

In certain embodiments, the delivery agent is formulated into a solid, a powder, a liquid, a gel, a capsule, a tablet, a pill, a pellet, an aerosol, or a particle.

A third aspect of the present invention provides a pharmaceutical composition comprising the delivery agent as described herein and at least one pharmaceutically acceptable excipient or pharmaceutically acceptable carrier.

In certain embodiments, the pharmaceutical composition is an orally administrable composition.

In certain embodiments, the pharmaceutical composition is an aerosolized composition.

A fourth aspect of the present invention provides a use of the delivery agent described herein in preparation of a pharmaceutical composition for improving bioavailability of a drug through inducing an M-cell GP2-mediated transcytosis in a subject in need thereof.

In certain embodiments, the delivery agent serves as a pharmaceutically acceptable carrier, vehicle, excipient, adjuvant, or additive.

In certain embodiments, the delivery agent serves as an immunomodulator or active ingredient for preventing, pre-treating, and treating diseases or cancers in the subject.

In certain embodiments, the pharmaceutical composition also comprises a drug other than the delivery agent.

In certain embodiments, the pharmaceutical composition is formulated into an orally administrable form.

In certain embodiments, the pharmaceutical composition is formulated into a solid, powders, a liquid, a gel, a capsule, a tablet, a pill, a pellet, or particles.

In certain embodiments, the pharmaceutical composition is formulated into an aerosolized form.

In certain embodiments, the pharmaceutical composition is formulated into particles or aerosols.

In certain embodiments, the drug comprises a small molecule, a peptide, an antibody, an antibody fragment, or a vaccine.

In certain embodiments, the M-cell GP2-mediated transcytosis is induced at small intestine mucosa or lung mucosa of the subject, and the subject comprises a human and a mammal other than a human.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in the corresponding Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the invention and apply throughout. Unless otherwise defined, all other technical terms used herein have the same meaning as commonly understood to one of ordinary skill in the art to which the invention belongs.

Other aspects and advantages of the invention will be apparent to those skilled in the art from a review of the ensuing description.

### BRIEF DESCRIPTION OF DRAWINGS

The above and other objects and features of the present invention will become apparent from the following description of the invention, when taken in conjunction with the accompanying drawings, in which:

FIG. 1 shows antitumor activity and immune responses induced by orally administrated RAP: (A) Timeline of 4T1 tumor-bearing mouse model. RAP was pre-treated for 7 d before 4T1 cells were implanted into the mammary fat pads of BALB/c mice or nude mice (n=10 for each group). Mice were sacrificed for collecting samples after 21 d treatment of RAP; (B and C) RAP-treated BALB/c mice exhibit smaller tumor size and weight; (D and E) RAP-treated nude mice exhibit similar tumor size and weight with no difference; (F) CD8+ T cells (CTL) were efficiently increased in tumors isolated from RAP-treated BALB/c mice; (G) CD4+CD25+ T cells (Treg) were significantly decreased in tumors isolated from RAP-treated BALB/c mice; (H) Total F4/80+ macrophages were increased in tumors of the RAP-treated group. M1 type macrophages (MTM, F4/80+CD11b^{high} macrophages) were induced by RAP, and Tumor-associated macrophages (TAM, F4/80+CD11^{blow} macrophages) were efficiently depleted by RAP in BALB/c mice; (I-L) Cytokines production in tumors isolated from BALB/c mice, including (I) IL-10, (J) IFN-γ, (K) MCP-1, and (L) TGF-β. Data are shown as mean ±SD. Significant difference *p<0.05, ** p<0.01, *** p<0.001, ns=no significance.

FIG. 2 shows different macrophages in tumor tissues induced by RAP: (A) F4/80+CD11b+ macrophages in 4T1 breast tumor-bearing BALB/C mice were analyzed by flow cytometry; (B) F4/80+CD11b+ macrophages and (C) percentage of macrophages in 4T1 breast tumor-bearing nude mice were analyzed by flow cytometry. M1 type macrophages (MTM), F4/80+CD11b^{high} macrophages. Tumor-associated macrophages (TAM), F4/80+CD11b^{low} macrophages.

FIG. 3 shows distribution of polysaccharides in bone marrow. Confocal images of bone marrow cells collected from mice treated with oral administrated FITC-RAP. PE-F4/80 was used to label macrophages, and FITC-RAP are captured by F4/80+ macrophages.

FIG. 4 shows high-performance gel permeation chromatography of polysaccharides and polysaccharides labeled with fluorescein isothiocyanate isomer I (FITC): (A) High-performance gel permeation chromatography coupled with fluorescence detector (HPGPC-FLD) chromatograms of Radix Astragali Polysaccharide (RAP) and FITC-RAP; (B) HPGPC coupled with Charged Aerosol Detector (CAD) chromatograms of FITC-RAP and RAP; (C) HPGPC-FLD chromatograms of Dendrobium officinale polysaccharide DOP and FITC-DOP; (D) HPGPC-CAD chromatograms of FITC-DOP and DOP.

FIG. 5 shows dynamic distribution of labeled RAP in the digestive tract and major organs after gavage: (A) Fluorescence images of major organs, including stomach, small intestine, caecum, colon, liver, kidneys, spleen, and mesenteric lymph nodes (MLN), collected from normal mice (n=6 per group) at 0 to 5 h after gavage with FITC-RAP (100 mg/kg); (B) the Fluorescence intensity of stomach, caecum, and colon; (C) HPGPC-FLD chromatograms of FITC-RAP in the caecum; (D) HPGPC-FLD chromatograms of FITC-RAP in the colon; (E) HPGPC-FLD chromatograms of FITC-RAP in the serum; (F) Fluorescence intensity of liver, MLN, kidneys, and spleen. Data are shown as mean±SD.

FIG. 6 shows transportation of intact polysaccharides into Peyer's patches (PPs) to initiate immune responses: (A) Fluorescence intensity and (B) HPGPC-FLD chromatograms of the small intestine contents collected from normal mice at 0 to 5 h after gavage with FITC-RAP (100 mg/kg); (C) Dynamic carbohydrate contents in the small intestine collected from normal mice at 0 to 5 h after gavage with unlabeled RAP (10 mg/kg) as detected by the phenol-sulfuric acid method; (D) Fluorescence images and (E) fluorescence intensity of PPs separated from the small intestines collected from normal mice at 0 to 5 h after gavage with FITC-RAP and FITC-DOP (100 mg/kg); (F) HPGPC-FLD chromatograms of PPs at 2 h after gavage with FITC-RAP (100 mg/kg); (G) Signaling pathways of NF-KB and MAPKs (p38 and ERK) of PPs at 1-4 h after oral administration with RAP (5 mg/mice), as determined by Western blotting assay. GAPDH is as control; (H) IL-6, (I) TGF-β, (J) IFN-γ, and (K) M-CSF productions in PPs of the small intestine collected from mice 24 hours after RAP treatment. PPs' homogenate was collected and detected by ELISA kits. Data are shown as mean±SD. Significant difference *p<0.05, ** p<0.01, *** p<0.001, **** p<0.0001, ns=no significance. Radix Astragali polysaccharide, RAP; Dendrobium officinale polysaccharide, DOP; High-performance gel permeation chromatography coupled with a fluorescence detector, HPGPC-FLD; fluorescein isothiocyanate isomer I, FITC.

FIG. 7 shows distribution of Dendrobium officinale polysaccharide DOP in Peyer's patches (PPs) and mesenteric lymph nodes (MLN): (A) HPGPC-FLD chromatograms of FITC-DOP in the PPs; (B) Fluorescence images of MLN, collected from normal mice (n=6 per group) at 0 to 6 h after gavage with FITC-DOP (100 mg/kg); (C) Fluorescence intensity of MLN; (D) HPGPC-FLD chromatograms of FITC-DOP of MLN. Data are shown as mean±SD. ns=no significance.

FIG. 8 shows uptake and transcytosis of intact RAP into PPs by M cells: (A) Images of whole mounts of PPs collected from mice in a ligated intestinal loop mouse model after FITC-RAP treatment using IVIS Lumina XR Small Animal Imaging System; (B) Fluorescence intensity of the region of interest (ROI) of the ex vivo images taken above; (C) HPGPC-FLD chromatograms of PPs with and without the FITC-RAP treatment for 1 h; (D) Ratio of the FITC-RAP uptake between intestinal epithelial cells (IECs) and GP2+M cells of PPs. M cells and IECs were isolated from PPs and small intestine segments without PPs of FITC-RAP (100 mg/kg)-treated mice, respectively; (E) After the ligated intestinal loop assay with FITC-RAP, PPs were stained with anti-GP2 mAb and DAPI. Uptake of FITC-RAP by GP2+M cells (Layer 1 to 9); (F) Serial X-Y sections of several GP2+M cells are shown from apical (image 1) to basal (image 9) domains; (G) Three-dimensional image of (F); (H) X-Z image of several M cells indicated that FITC-RAP were transported into the basal layer of GP2 receptors.

FIG. 9 shows design and validation of the in vitro human M cell model: (A) Protocol used to establish the in vitro M cell model and its application; (B) Confocal microscopic images of co-culture transwell inserts were stained with GP2 mAb and subsequently with Alexa 647-conjugated secondary antibody. DAPI was used as a DNA-specific stain; (C) Flow cytometry analysis of nanoparticles in the supernatant collected from the lower layer of mono-culture and co-culture groups (n=3); (D) Percentage of nanoparticles transported by the M cell model. Data are shown as mean±SD. Significant difference **** p<0.0001.

FIG. 10 shows transportation of intact RAP in an in vitro model of the human intestinal follicle-associated epithelium (FAE): (A) Confocal microscopic images of co-culture transwell inserts treated with FITC-RAP and then stained with GP2 mAb and subsequently with Alexa 647-conjugated secondary antibody. DAPI was used as a DNA-specific stain. These images show that FITC-RAP was internalized by GP2+M cell; (B) Flow cytometry analysis of FITC-RAP in the supernatant collected from lower layers of mono-culture and co-culture groups (n=9 per group); (C) HPGPC-FLD chromatograms of FITC-RAP in the supernatants collected from lower layers of mono-cultures and co-cultures (n=9 per group); (D) IL-6 and (E) IL-12 secretion of RAW264.7 cells treated with supernatants collected from the lower layer of mono-cultures and co-cultures (n=9 per group) 2 h after loading 200 μl of RAP (2 mg/ml), compared with cytokines produced from RAW 264.7 cells treated with RAP at different concentration (0.1, 1, 10, and 100 mg/ml). Data are shown as mean±SD. Significant difference *p<0.05, ** p<0.01, *** p<0.001, **** p<0.0001, ns=no significance.

FIG. 11 shows direct contact of M cell-transported RAP with follicle dendritic cells (FDCs) in Peyer's patches: (A) Confocal images of PPs frozen sections (dome zone), collected from the ligated intestinal loop assay treated with FITC-RAP, and stained with GP2, CD11c, and DAPI. Three-dimensional image of PP indicated that FITC-RAP was transported into sub-epithelial dome (SED) and contacted CD11c+ dendritic cells: (B) Confocal images of PPs frozen sections (subepithelial zone) in PPs.

FIG. 12 shows that GP2 binds to RAP to facilitate its transcytosis: (A) Frozen section of PPs collected from the ligated intestinal loop assay stained with GP2 and DAPI. It is demonstrated that FITC-RAP was transported by GP2+M cells into PPs; (B) Serial X-Y sections of PPs FAE with GP2+M cells from up-layer (image 1) to down-layer (image 10) domains of the frozen PPs sections. PPs were collected from the ligated intestinal loop assay with FITC-RAP treatment and stained with GP2 and DAPI. Three-dimensional image of PP indicated that FITC-RAP was binding with GP2 and internalized from the apical to the basal cells.

FIG. 13 shows targeted disruption of mouse GP2 gene: (A) Genotyping strategy of targeting the gene. Wild-type band is 574 bp; mutated band is 557 bp. +/+, wild-type; +/−, heterozygous mouse; −/−, homozygous mouse; (B) Southern blot analysis of offspring from heterozygote intercrosses.

FIG. 14 shows M cells in the small intestine by recognizing the specific markers GP2 and NKM 16-2-4: (A) Confocal microscopic images of whole-mount PPs stained with DAPI. Dome zone, which is the location of FAE in PPs, is marked by a dotted white circle; (B-D) Confocal microscopic images of whole-mount PP domes stained with anti-NKM 16-2-4 mAb and anti-GP2 mAb and subsequently with Alexa 488-labelled and Alexa 647-labelled secondary antibody. DAPI was used as a DNA-specific stain. For a three-dimensional image of PP whole-mount staining; (E) Confocal microscopic images of frozen PP tissue isolated from wild BALB/c mice stained with anti-NKM 16-2-4 mAb and anti-GP2 mAb and subsequently with Alexa 488-labelled and Alexa 647-labelled secondary antibody. DAPI was used as a DNA-specific stain; (F) Cryosections of ileal PPs stained with H&E and visualized by light microscopy. Abbreviations: FAE, follicle-associated epithelium; SED, sub-epithelial dome; LF, lymphoid follicle.

FIG. 15 shows that GP2 mediates M cell's transcytosis of RAP: (A) Ex vivo images of PPs collected from the ligated intestinal loop assay stained with anti-NKM 16-2-4 mAb and subsequently with Alexa 647-labeled secondary antibody and DAPI; (B) PPs collected from wild type (WT) C57 mice, WT-model mice, GP2^{-/-} mice, and GP2^{-/-} model mice (n=20 per group) were imaged in the ligated intestinal loop assay using an IVIS Lumina XR Small Animal Imaging System; (C) Fluorescence intensity values of ROI of the above ex vivo images; (D) HPGPC-FLD chromatograms of PPs (n=20 per group) with or without the treatment of FITC-RAP for 1 h; (E, F, G, H, I, and J) After oral administration of RAP (100 mg/kg) in WT (GP2^{+/+}) mice and GP2^{-/-} mice (n=10 per group) for 24 h, GP2^{-/-} mice failed to induce the differentiation of (E) CD11c+DCs, the maturation of (F) CD11c+CD80+ DCs, the activation of (G) CD11c+TLR4+DCs, and upregulation production of (H) IL-6 and (I) TNF-α, and the inhibition of (J) IL-10. Data are shown as mean±SD. Significant difference *p<0.05, ** p<0.01, ** p<0.001, **** p<0.0001, ns=no significance.

FIG. 16 shows GP2+M cells in epithelial cells from small intestine segments without Peyer's patches, Peyer's patches of WT mice, and Peyer's patches of GP2^{-/-} mice. Cells were stained with PE-GP2 antibody. Data are shown as mean±SD.

FIG. 17 shows flow analysis of immunes cells of PPs isolated from WT (GP2^{+/+}) mice and GP2^{-/-} mice, after oral administration of RAP (100 mg/kg) for 1 day (D): (A) Percentage of activated CD11c+ dendritic cells; (B) Percentage of CD11c+MHCII+ DCs. Data are shown as mean±SD.

FIG. 18 shows flow analysis of immunes cells of PPs isolated from WT (GP2^{+/+}) mice and GP2^{-/-} mice, after oral administration of RAP (100 mg/kg) for 1 day (D): (A) Percentage of CD11c+CD80+ DCs; (B) Percentage of CD11c+TLR4+ DCs. Data are shown as mean±SD.

FIG. 19 shows transportation of RAP by M cells of human ileum PPs: (A) Section of human distal ileum with PPs indicated by a black arrow; (B) An enlarged view of a PP is noted by a black arrow (2.5×). The tissue section was cut from the distal ileum in FIG. 5(A); (C) Confocal microscopic images of the whole-mount PP domes stained with anti-GP2 mAb and subsequently with Alexa 647-labelled secondary antibody. DAPI was used as a DNA-specific stain. The dome zone, which is the location of FAE in PPs, is marked by a dotted white circle; (D) Confocal microscopic images of the whole-mount PP domes stained with anti-human GP2 antibody and DAPI, showing that FITC-RAP was bound to GP2+M cells; (E) Three-dimensional confocal microscopic images of FAE. FITC-RAP was internalized by human GP2+M cells; (F) Confocal microscopic image of frozen sections of human ileal PPs stained with DAPI. FITC-RAP was found in the SEM of PPs. Abbreviations: FAE, follicle-associated epithelium; SED, sub-epithelial dome; LF, lymphoid follicle.

### DETAILED DESCRIPTION OF THE INVENTION

The following examples are provided to demonstrate how Radix Astragali Polysaccharide (RAP), a TCM-derived macromolecule with an average molecular weight of 1334 kDa composed of Rha, Ara, Glc, Gal and GalA in a molar ratio of 0.03:1.00:0.27:0.36:0.30, and a backbone consisting of 1,2,4-linked Rhap, α-1,4-linked Glcp, α-1,4-linked GalAp6Me, β-1,3,6-linked Galp, with branches at O-4 of the 1,2,4-linked Rhap and O-3 or O-4 of β-1,3,6-linked Galp; sidechains being mainly α-T-Araf and α-1,5-linked Araf with O-3 as branching points, having trace Glc and Gal; and terminal residues being T-linked Araf, T-linked Glcp and T-linked Galp (Yin et al., Separation, structure characterization, conformation and immunomodulating effect of a hyperbranched heteroglycan from Radix Astragali. Carbohydrate Polymers, 87(1), 667-675), triggers antitumor immunity after oral dosing. In other embodiments, the RAP can have an average molecular weight ranging from 100 to 1500 kDa comprising Rha, Ara, Glc, Gal and GalA in a molar ratio of 0.01-0.03:1.00:0.20-10.00:0.01-0.50:0.01-0.30, wherein a backbone thereof includes 1,2,4-linked Rhap, α-1,4-linked Glcp, α-1,4-linked GalAp6Me, or β-1,3,6-linked Galp; and one more sidechains include α-T-Araf, α-1,5-linked Araf with O-3, T-linked Araf, T-linked Glcp, and T-linked Galp. RAP may form a chemical conjugate such as a covalent conjugate with a drug to be delivered or an admixture therewith. RAP may also be formulated as a drug delivery agent or composition to be administered in conjunction with one or more drugs or pharmaceutical composition comprising thereof to be delivered. Apart from oral dosing, RAP may also be formulated as aerosols or aerosolized composition comprising thereof to be administered via inhalation or respiratory route of administration. An immunity-dependent antitumor effect could be attributed to RAP-induced rapid immune responses in Peyer's patches (PPs). Comprehensive evidence in vivo and in vitro using mice and human gut explants revealed that RAP is transported from the gut lumen to follicular dendritic cells (FDC) by transcytosis through microfold (M) cells. Other than via an intestinal mucosa of the gut lumen, M cells are also found in lung mucosa, and therefore RAP may also be considered capable of triggering the same immunity-dependent antitumor effect in the respiratory tract as that in the gut lumen of a subject which can be a human or non-human mammal. Comparison with control polysaccharides revealed that this transport pathway was selective, and loss of transport in GP2^{-/-} mice established GP2 as the transport receptor. M cell-mediated transcytosis of RAP was also verified in human subjects. These results solve the longstanding delivery paradox for medicinal polysaccharides and offer a new targeting strategy for oral delivery of macromolecules more generally.

### EXPERIMENTAL SECTION

### Human Subjects, Animals, and Cells

For the study of human subjects, it was performed in accordance with the established ethical guidelines and approved (2019-KY-373) by the research ethics committee of the School of Medicine, Zhengzhou University, Zhengzhou, China.

BALB/C mice, nude mice and C57 BL/6 mice were purchased from the Chinese University of Hong Kong. GP2-heterozygous (GP2^{-/+}) mice (C57 background) were obtained from Cyagen Biosciences (Guangzhou, China). GP2-deficient (GP2^{-/-}) mice were obtained by crossing the GP2^{-/+} mice and identified by PCR. Five-to eight-week-old mice were used in this study. All animal experiments followed the Animals Ordinance guidelines, Department of Health, Hong Kong SAR ((16-65) in DH/HA&P/8/2/6, (19-151) in DH/HT&A/8/2/6). Caco-2 cells, Raji B cells, and RAW264.7 cells were obtained from American Type Culture Collection (ATCC).

### Antibodies and Reagents

PE-conjugated rabbit anti-mouse glycoprotein 2 (GP2) antibody (2F11-C3), rat anti-mouse GP2 antibody (2F11-C3), and mouse anti-human GP2 antibody (3G7-H9) were purchased from MBL International. Rat anti-mouse NKM 16-2-4 antibody and APC-conjugated rat anti-mouse NKM 16-2-4 antibody was obtained from Merck. CD11c primary antibody, goat anti-rabbit or rat IgG antibody conjugated with Alexa Fluor 647, and goat anti- rat IgG antibody conjugated with Alexa488 were purchased from Abcam. Flow cytometry antibodies, including CD11c-PE, MHCII-PerCP, CD80-FITC, and TLR4-FITC, were bought from BioLegend. Phenol-sulfuric acid, 4′,6-diamidino-2-phenylindole DAPI, and fluorescein isothiocyanate isomer I FITC, and methyl sulphoxide were obtained from Merck. FLUORESBRITE® Multifluorescent Microspheres 0.20 μm were purchased from Polysciences. All the human and mouse ELISA kits were purchased from eBioscience. All primers used for PCR were synthesized by BGI-Shenzhen.

### 4T1 Breast Tumor Mouse Model and Treatment

Five-week-old Balb/c female mice and nude female mice were used for tumor model (8-10 mice/group). Before the implantation of tumor cells, RAP-treated group or fecal microbiota transplant (FMT)-treated group were pre-treated for one week. Stools used in FMT was collected from mice in control (H₂O) and RAP-treated group and performed according to an established protocol. The implantation of 4T1 cells were performed according to previous study. Briefly, 4T1 cells (2×10⁴ mice) were implanted with 27-G needle. When tumors begin to develop, vernier caliper was used to measure tumor diameters and calculate tumor volume. Three weeks later from the development of tumor, animals were sacrificed and interested samples were harvested for investigation, including measurement of tumor weight and detection of immune cells and cytokines in tumor and immune system.

### Preparation of Polysaccharides and FITC Labeled Polysaccharides

RAP was prepared from the water extract of the dried roots of Astragalus membranaceus. It was labeled with fluorescein isothiocyanate isomer I (FITC) as reported. Briefly, RAP (1.0 g) was dissolved in methyl sulphoxide (8 mL) containing a few pyridine drops. The FITC (80 mg) was added to the RAP solution, followed by dibutyltin dilaurate (16 μL). The mixture was heated for 2 h at 95° C. After repeated precipitation in ethanol (90% v/v) to collect the precipitate and remove the free dye, the FITC-RAP was further re-dissolved in water and further purified by molecular sieve (3 kDa cut-off). Control polysaccharide DOP was prepared from Dendrobium officinal Caulis and labeled with FITC as reported previously.

### Dynamic Distribution of FITC-RAP in the Gastrointestinal System of Mice

FTIC-RAP (10 mg in 1 mL distilled water) was orally administrated to each mouse (n=6). After the mice were sacrificed at 0, 1, 2, 3, 4, and 5 h after oral administration, blood and major tissues, including liver, spleen, kidney, stomach, small intestine, mesenteric lymph nodes (MLN), Peyer's patches (PPs), caecum, and colon, were collected and imaged using an IVIS Lumina XR in vivo imaging system (PerkinElmer) immediately. The molecular size of FITC-RAP was monitored using high-performance gel-permeation chromatography. For Western blotting (WB), PPs from different groups were collected at other time points (0, 1, 2, 3, and 4 h after oral administration) and prepared for WB. For ELISA assay, PPs were collected 24 hours after RAP treatment, then homogenized and centrifugated at 15,000 rpm for 10 min to obtain the supernatant for cytokine production assessment using ELISA kits. Similarly, the dynamic distribution of FITC-DOP control in MLN and PPs was investigated.

Dynamic Distribution of Polysaccharides Analyzed by High-Performance Gel Permeation Chromatography Coupled With Fluorescence Detector (HPGPC-FLD) Analysis

The tissues collected from the above dynamic distribution analysis were homogenized using a 3-fold volume of 0.1 mol/L phosphate buffer (pH 7.4) and centrifuged at 15,000 rpm for 10 min. Especially, the PP collected from the ligated loop assay model were homogenized using 200 μL PBS and centrifuged at 15,000 rpm for 10 min. The supernatant was collected and stored at −20° C. for chromatographic analysis. The separation was achieved on a TSK GMPWXL column (164× 7.8 mm i.d., 10 μm) system operated at 40° C. using an Agilent-1100 HPLC system equipped with FLD. Ammonium acetate aqueous solution (20 mM) was used as a mobile phase at a flow rate of 0.6 mL/min. The excitation wavelength and emission wavelength of FLD were 495 and 515 nm, respectively.

### Western Blotting

PPs collected at different time points (0, 1, 2, 3, and 4 h), were lysed with RIPA protein extraction reagent containing protease and phosphatase inhibitors for 30 min. Protein samples were separated by 10% SDS-PAGE and then transferred to a PVDF membrane. Membranes were blocked in 5% blocker milk (BioRad) at RT (i.e. 20° C.-25° C.) for 1 h then incubated with primary antibodies at 4° C. overnight with shaking. The primary antibodies were GAPDH, P38, p-P38, P65, p-P65, ERK and p-ERK, according to our previous study. The membranes were washed three times with PBST (0.1% Tween 20) and incubated with horseradish peroxidase (HRP)-conjugated secondary antibodies for 1 h. Protein bands were visualized using enhanced chemiluminescence (ECL) detection reagent and medical X-ray film.

### ELISA Assay for Cytokines

The supernatants collected from cell culture or the homogenates of PPs were centrifuged at 3000 g for 10 min. According to the manufacturer's instructions, cytokines IL-6, IL-10, IL-12, TNF-α, M-CSF, TGF-β, MCP-1, IFN-γ, VEGF, and MMP-9 were determined using ELISA kits

### Carbohydrates Detection by Phenol-Sulfuric Acid Method

The intestinal contents of BALB/C mice that had received oral doses of RAP (100 mg/kg/mice) were collected at 0, 1, 2, 3, 4, 5 h after oral administration (n=6 each group) for carbohydrates detection by the phenol-sulfuric acid method. In brief, intestinal contents collected from the stomach, the small intestine, and the large intestine of mice treated with and without RAP were homogenized and centrifuged at 15000 rpm for 10 min. 50 μl of each supernatant in a well of 96-well microplate was maximumly mixed with 150 μl of concentrated sulfuric acid rapidly. 30 μl of 5% phenol in water was then added into the mixed solution and incubated for 5 min at 90° C. in a static water bath. The plate was then cooled to RT and wiped dry to detect at A490 nm by a microplate reader. Glucose was used as a reference standard to establish standard curves. The experiments were repeated three times.

### Ligated Intestinal Loop Assay

C57BL/6 wild-type (WT) mice, and GP2^{-/-} C57BL/6 mice were anesthetized using isoflurane. To prepare ligated intestinal loops, incision was made near the linea alba of the abdomen to expose the small intestine. FITC-RAP (10 mg/mL) in 100 μl PBS was injected into the ligated intestinal loop. The mice were kept under anesthesia for 1 h after the injection. PPs were excised and rinsed with PBS 5 times. The cleaned PPs were photographed using an IVIS Lumina XR and then processed for HPGPC-FLD analysis, whole-mount staining, sectioning, and further microscopic analysis.

### Immunofluorescence Staining and Confocal Microscopy

Frozen sections of PPs from the ligated loop assay were washed three times with PBS and blocked with 5% normal goat serum in PBS for 1 h. Sections were incubated with anti-mouse GP2, anti-mouse NKM 16-2-4 antibody, or anti-mouse CD11c antibody overnight at 4° C. PPs sections were washed three times with PBS and then treated with Alexa Fluor647 secondary goat anti-rabbit, Alexa Fluor5-conjugated anti-rat antibodies for 1 h at RT, followed by three consecutive PBS washes. The cell nuclei were stained with 1 μg/mL 4′,6-diamidino-2-phenylindole (DAPI) for 15 min. Sections were washed three times with PBS and mounted with an anti-fade mounting medium. Images were captured with a Leica TCS SP8 confocal laser scanning microscope.

### Whole-Mount Staining and Confocal Microscopy

For whole-mount staining, mouse PPs were isolated from the small intestine and washed 3 times with 1×ice-cold PBS. The PPs were fixed in 4% paraformaldehyde overnight at 4° C. and then permeabilized with 0.1% Triton X-100 for 30 min. PPs were blocked with 5% normal goat serum in PBS for 1 h. A primary rat anti-mouse glycoprotein 2 (GP2) and 1 μg/ml DAPI in PBS were used to incubate PPs for 1 h at RT, followed by rinsing 3×5 mins with PBS. The second goat anti-rat IgG antibody conjugated with Alexa647 was further stained for 1 h at RT. PPs were washed 3×5 mins with PBS and mounted with the anti-fade reagent. Images were analyzed and captured by a confocal laser microscope (SP8, Leica Microsystems).

### Flow Cytometry Analysis

For the detection of RAP-induced immune responses, tumor tissues and PPs were minced into small pieces and grounded with a syringe plug. For the isolation and identification of M cell, PPs and small intestine segments collected from FITC-RAP-treated mice were incubated in PBS with 0.5 mM EDTA and stirred at 37° C. for 20 min. For the isolation and detection of dendritic cells, PPs collected from WT mice and GP2--mice were ground with a syringe plug. Tissues and cells were collected and rinsed with ice-cold PBS at 400 g for 5 min, followed by filtering through a 70 μm filter. Single cell suspensions were prepared for antibody staining. Cells were incubated with antibodies or the matching isotypes for 25 min at RT. The stained cells were rinsed twice and re-suspended in PBS and analyzed by FACSAria III (BD Biosciences). Data analysis was performed with FlowJo V10 software.

### In Vitro M-Cell-Like Model

Caco-2 cells were grown in flasks in 10% (v/v) fetal calf serum DMEM at 37° C. under a 10% CO₂ water-saturated atmosphere. Caco-2 cells were grown on Transwell polyester inserts (0.3 μm pore size, 12 mm diameter, Corning Costar) coated with 100 μg/mL matrigel basement membrane matrix phenol red-free (Becton Dickinson), prepared in pure cold DMEM without phenol red. Raji B cells (5× 10⁵) were resuspended in DMEM and added to the basolateral chamber of 7-day-old Caco-2 cell monolayers. The co-cultures were maintained for 5 days. Mono-culture of Caco-2 cells, cultivated as above but without the Raji B cells, were used as control. Inserts were used for all the following experiments, including assessing M cell functionality by nanoparticle transport measurement using flow cytometry, M cell demonstration in co-cultured monolayers using an immunofluorescence staining assay, and determination of transported RAP using chromatographic analysis and ELISA assay. The transportation function of this in vitro M cell model was first validated using nanoparticle. FITC-RAP (2 mg/mL) in 400 μl DMEM were added to the upper layers of mono-culture and co-culture inserts and then incubated for 2 h at 37° C. under 5% CO₂. All inserts were removed, and lower solutions were collected for chromatographic analysis. The lower solution collected from the co-culture group treated with unlabeled RAP was further used to treat RAW264.7 cells in a 96-well plate for 24 h, and the supernatants were sampled for IL-6 and IL-12 detection using ELISA kits.

### Identification of M cells in Co-Cultured Monolayers

Three co-culture and mono-cultures inserts were used to detect the presence of M-like cells by immunofluorescence. All inserts were washed three times with PBS and fixed with 4% paraformaldehyde (PFA) for 15 min, and then blocked for 1 h in 5% of normal goat serum in PBS. Rat anti-human GP2 antibody was used as the primary antibody and incubated overnight at 4° C. After washing three times with PBS, all inserts were incubated with Alexa Fluor647-conjugated goat anti-rat secondary antibody for 1 h in the dark. DAPI was used to stain cell nuclei for 15 min in the dark. All inserts were washed three times with PBS, and the polyester membranes were cut and mounted using an anti-fade mounting medium. Slides were performed with a Leica TCS SP8 confocal laser scanning microscope.

### Study of Human Subjects

Twenty volunteers who needed surgery were enrolled in this study. The age of the participants ranged from 20 to 50 years. All distal ilea of the small intestine were collected after their surgeries. Twenty samples of the small intestine with PP were used in this study. In brief, the small intestines with PPs were stored in ice-cold PBS with 5% FBS after surgery. The small intestine segment was ligated with a surgical suture for the ligated loop assay. It then was injected with 10 ml FITC-RAP solution (5 mg/ml) followed by incubation at 37° C. under 5% CO₂ for 2 h. PPs were collected for whole-mount staining, preparation of frozen sections, and immunofluorescence staining.

### Statistical Analysis

GraphPad Prism 7.0 software was used to assess statistical significance. Statistical analysis of data was carried out using standard t-tests, one-way ANOVA followed by Tukey's post hoc analysis, and two-way ANOVA followed by Bonferroni post hoc analysis. Statistical significance was determined at *p<0.05, ** p<0.01, *** p<0.001, **** p<0.0001. All data are expressed as mean±SD. Each experiment was conducted three times or as indicated in the figure legends.

### RESULTS

### Immunity-Dependent RAP'S Anti-Cancer Activity

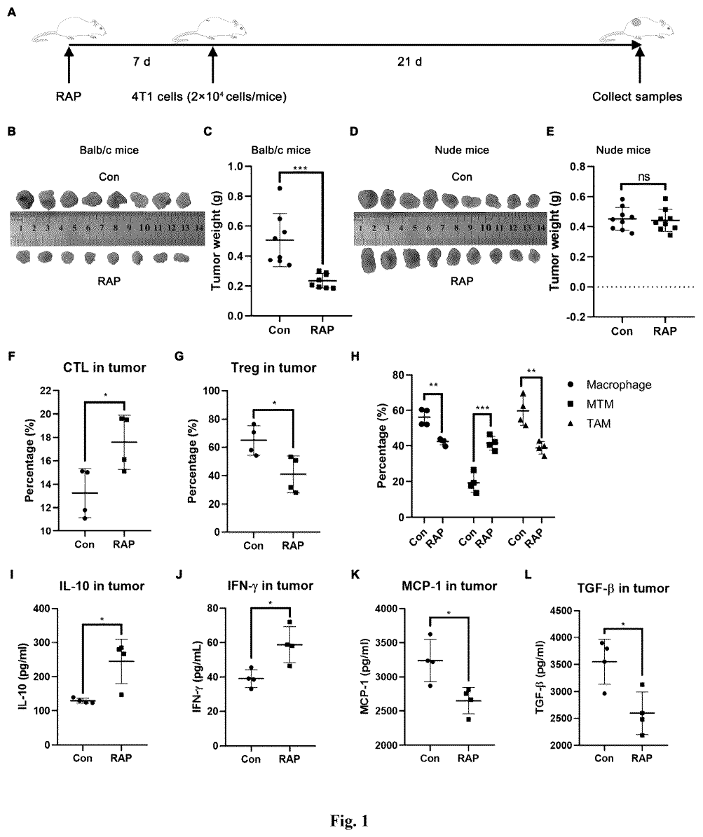

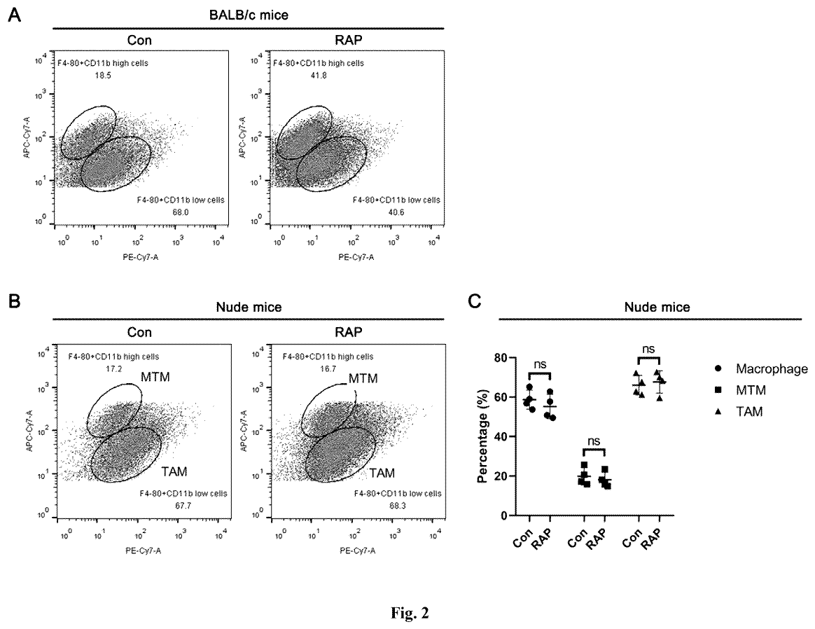

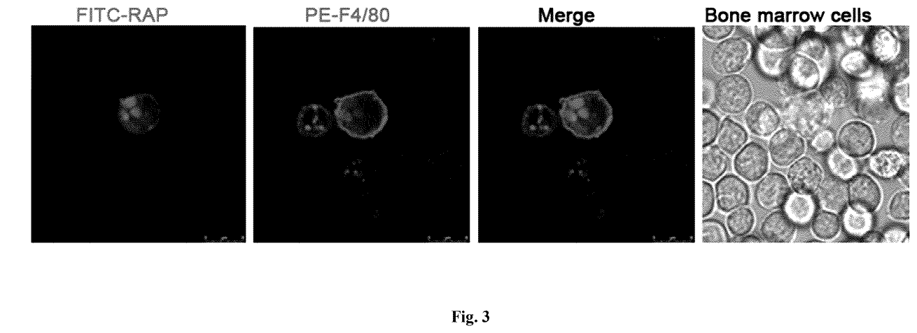

The diverse beneficial effects of Radix Astragali polysaccharide (RAP) were widely reported to be associated with its immunoregulation effects. The present inventors evaluated RAP's effects towards the tumor growth and related immune responses in tumor tissue on a 4T1 tumor mice model (FIG. 1A). The results showed that RAP significantly suppressed the tumor growth (FIG. 1B and FIG. 1C) and induced antitumor immune responses in tumor tissues, including the increase of CD3+CD8+ cytotoxic T lymphocyte (CTL) (FIG. 1F), the decrease of CD4+CD25+ regulatory T cells (Treg, FIG. 1G) and F4/80+ macrophages (FIG. 1H), the differentiation from F4/80+CD11b^{low} tumor-associated macrophage (TAM) to F4/80+CD11b^{high} M1 type macrophages (MTM) (FIG. 1H and FIG. 2A), and the changes of antitumor-related cytokines IL-10 (I), IFN-γ (J), MCP-1 (K), and TGF-β (L). The present inventors further tested RAP's antitumor effects on immunodeficient nude mice and neither the tumor suppression (FIG. 1D and 1E) nor the differentiation of macrophages in the tumor tissue (FIG. 2B and 2C) was observed in RAP-treated mice. All these above results suggest that RAP's anti-cancer activity is immunity-dependent.

### Intact RAP Is Specifically Delivered to Peyer's Patches (PPs) to Induce Immune Responses Directly

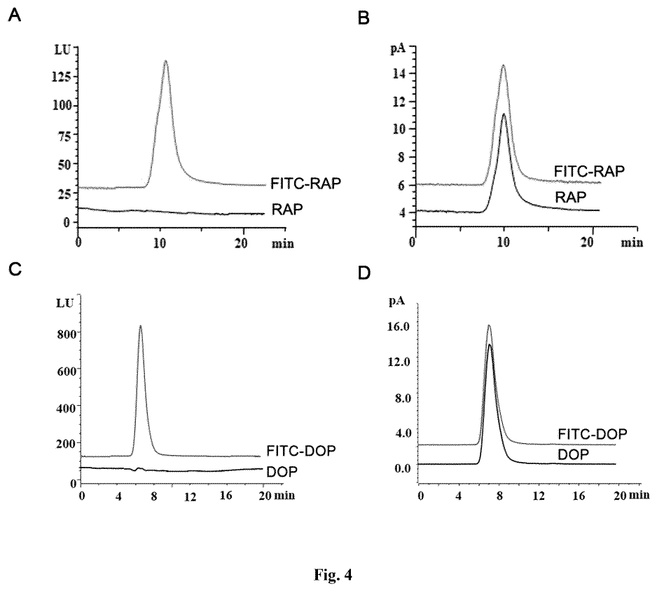

The present inventors' previous results (Bao et al., “Radix Astragali polysaccharide RAP directly protects hematopoietic stem cells from chemotherapy-induced myelosuppression by increasing FOS expression”, Int J Biol Macromol. 2021 Jul. 31;183:1715-1722. doi: 10.1016/j.ijbiomac.2021.05.120. Epub 2021 May 24. PMID: 34044030) showed that the positive signal of fluorescent-labeled RAP (FITC-RAP) were binding with CD34+ cells in the bone marrow. The present inventors further screened other immune cells in the bone marrow and found that F4/80+ macrophages could also bind with FITC-RAP (FIG. 3). This suggests that RAP is able to enter the host and directly induce the immune responses.

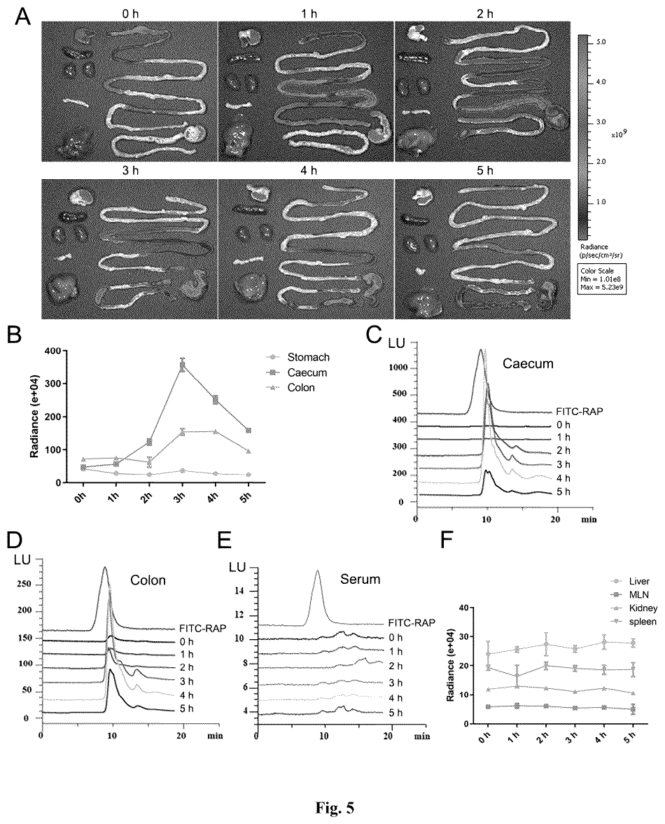

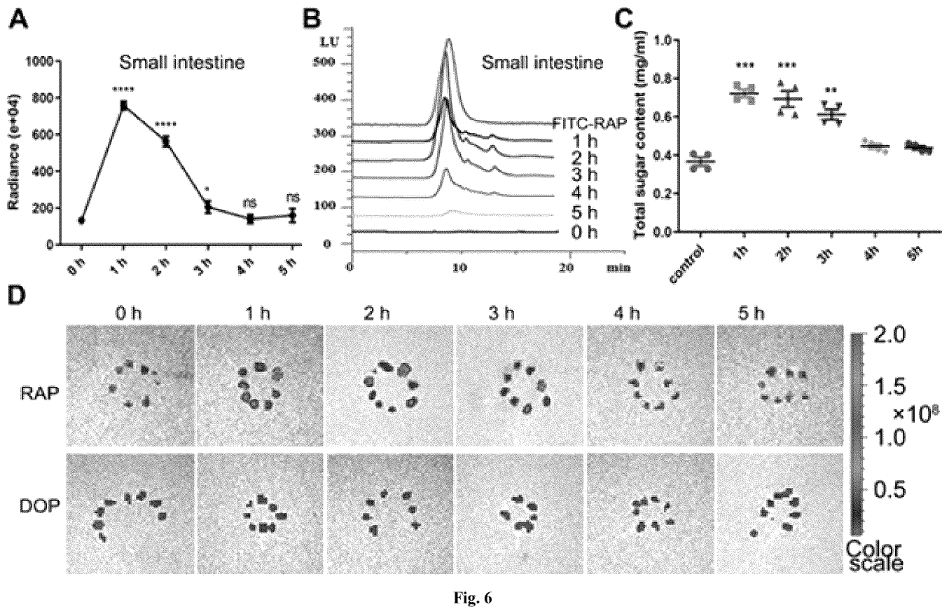

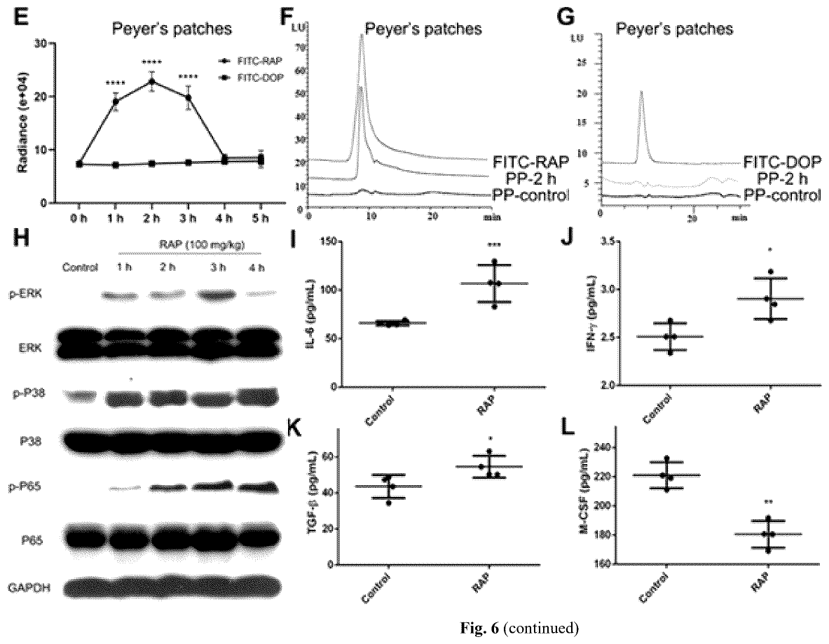

The intestinal mucosal immune system is the most likely-initiated part of the host immune system to be affected by oral administrated polysaccharides. Before investigating the connection between RAP and immune cells, the dynamic distribution of oral RAP in the host is the first thing to figure out. The present inventors' previous study (Li et al., “Destiny of Dendrobium officinale Polysaccharide after Oral Administration: Indigestible and Nonabsorbing, Ends in Modulating Gut Microbiota”, J. Agric. Food Chem. 2019, 67, 21, 5968-5977, https://doi.org/10.1021/acs.jafc.9b01489) showed that Dendrobium officinale polysaccharide (DOP) could not be delivered into the host, offering a perfect control polysaccharide here. To track the localization of orally administrated polysaccharides in mice, the present inventors firstly labeled RAP and DOP with fluorescein isothiocyanate isomer I (FITC). High-performance gel permeation chromatography showed that all the fluorescence signals were covalently bound to their respective polysaccharides (FIG. 4A-D). Whole imaging allowed visualization and quantification of the passage of labeled RAP through the gut (FIG. 5A-B). Gel permeation chromatography showed that labeled RAP remained intact in the small intestine for several hours (FIG. 6A-C) but immediately degraded in the caecum and colon (FIG. 5C-D). Measurement of fluorescence in the liver, kidney, spleen, mesenteric lymph nodes (MLN), and circulation showed no detectable increase (FIGS. 5E-F), confirming low oral bioavailability of macromolecule.

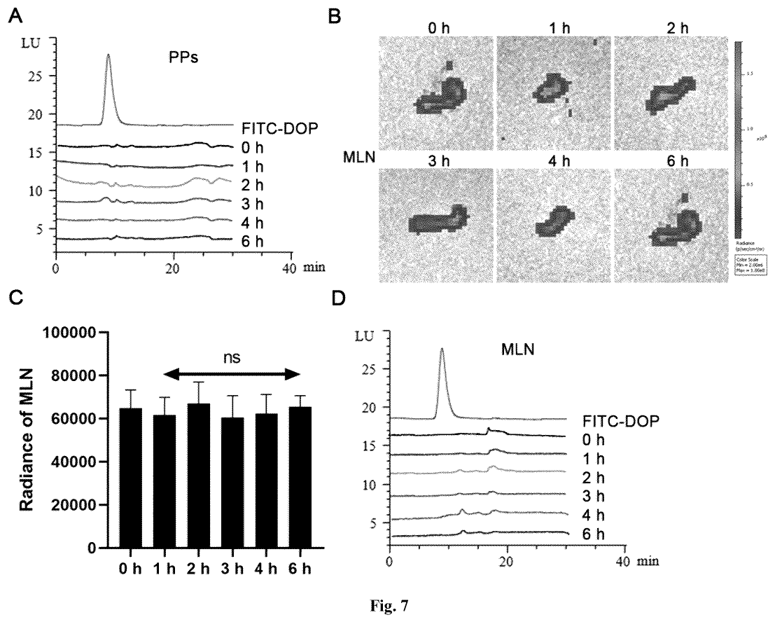

To measure the delivery of RAP to the mucosal immune system, the present inventors dissected and imaged PPs at different times after oral dosing. RAP, but not DOP, transiently accumulated in these areas where gut immune cells gather (FIG. 6D). Gel permeation chromatography showed that most of the RAP in PPs were undegraded (FIG. 6E), demonstrating the transport of the intact polysaccharide across the gut wall. Lack of DOP accumulation in PPs (FIG. 6D-E and 7A), or MLN (FIG. 7β-D), demonstrated that RAP transport was site-specific.

Expectedly, intact RAP was transported into PPs directly, offering an opportunity for RAP to contact immune cells directly. the present inventors next tested RAP's effects on immune responses in PPs by measuring key signaling pathways affected by RAP in vitro. Nuclear factor kappa-light-chain-enhancer of activated B (NF-κB), p38, and extracellular signal-regulated kinase (ERK) pathways were significantly activated by oral RAP in several hours (FIG. 6G), suggesting RAP-induced quick immune responses by entering PPs. Consistent with the present inventors' previous in vitro report (Wei et al., “TLR-4 may mediate signaling pathways of Astragalus polysaccharide RAP induced cytokine expression of RAW264.7 cells”, Journal of Ethnopharmacology, Volume 179, 2016, Pages 243-252, ISSN 0378-8741, https://doi.org/10.1016/j.jep.2015.12.060), it is found that cytokines IL-6, TGF-β, and IFN-γ were induced (FIG. 6H-J), and expression of M-CSF was inhibited (FIG. 6K). These results suggest that FITC-RAP rapidly enters PPs and triggers immune signaling that likely plays a central role in the therapeutic actions of RAP.

### M Cells Mediate Epithelial Transcytosis of Intact RAP

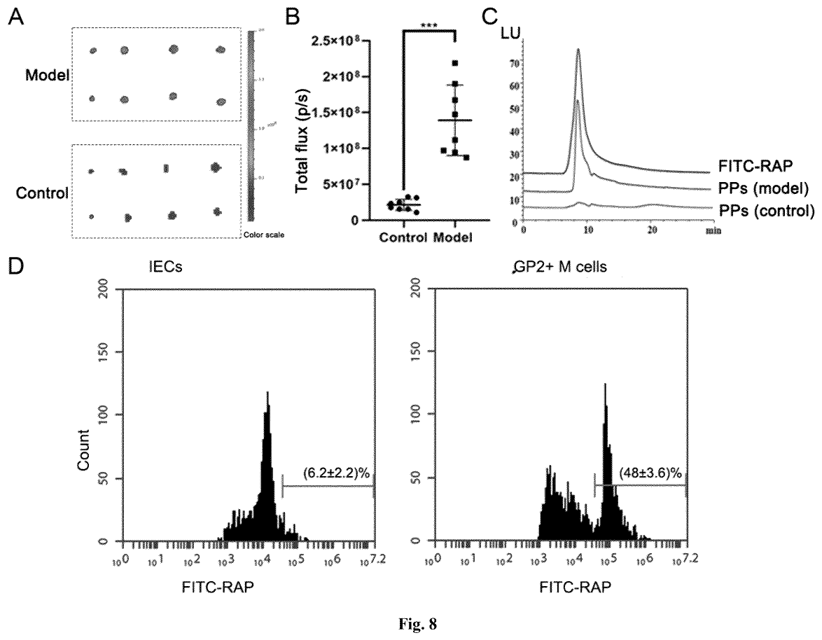

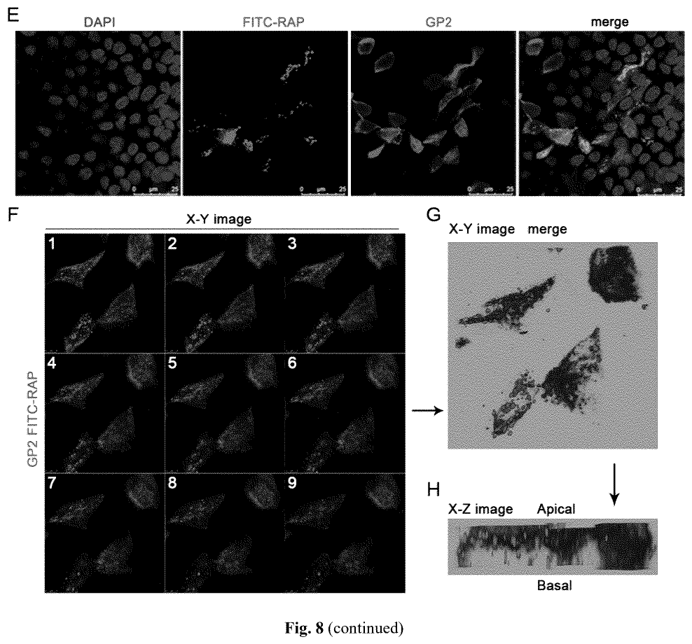

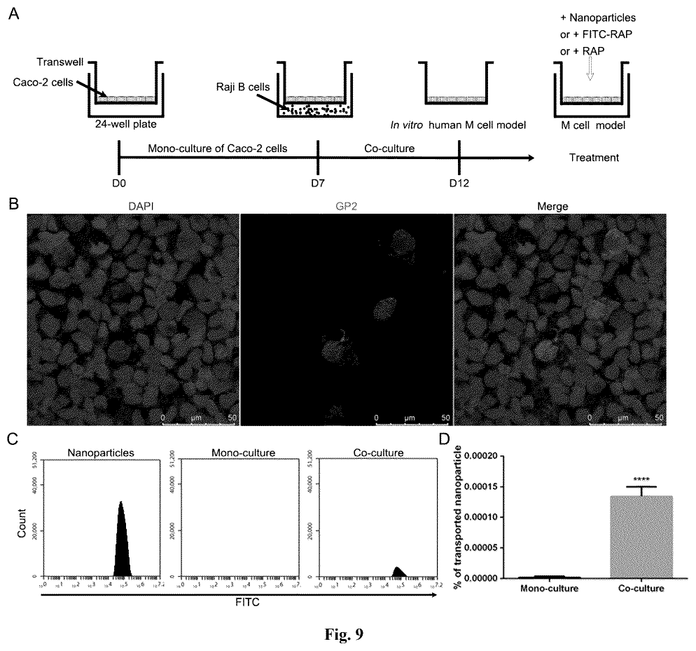

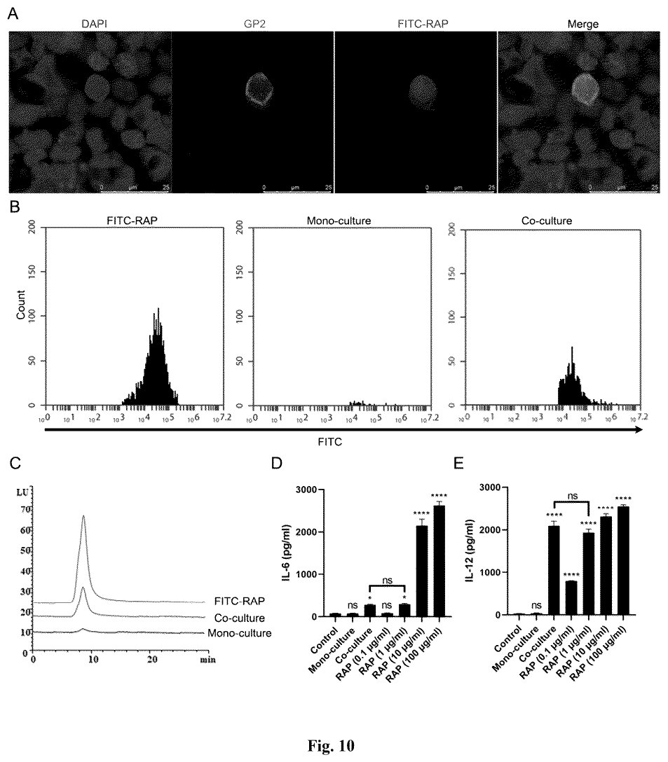

To assess how RAP is transported across the gut wall, the present inventors tracked FITC-RAP in a ligated intestinal loop mouse model of C57BL/6 wild-type mice. Application of a whole animal imaging system showed that 1 hr after injection into the gut loop, FITC-RAP concentrated in regions associated with PPs (FIG. 8A and 8B) where it was present in an undegraded form (FIG. 3C). Further flow cytometry analysis of gut cells indicated that GP2+M cells instead of other intestinal epithelial cells accumulated FITC-RAP in the ligated loop model (FIG. 8D). The specific transportation of FITC-RAP by GP2+M cells was further demonstrated by confocal microscopic images of fixed and stained whole-mount preparation (FIG. 8E-H) of PPs isolated from mice 1 h after FITC-RAP treatment. To verify the M cell-mediated polysaccharide transcytosis, the present inventors also established and validated an in vitro M cell-like model by co-culturing the human gut epithelium cell line Caco-2 with Raji B cells on filter inserts (FIG. 9A-D). Only after differentiation to M-like cells could the epithelial cells transport RAP into the lower layer of the inserts (FIG. 10A-E).

### M Cell-Mediated Transcytosis of RAP Is GP2 Receptor-Dependent

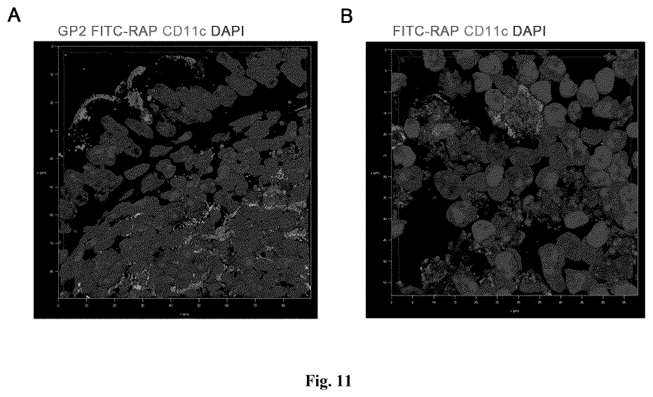

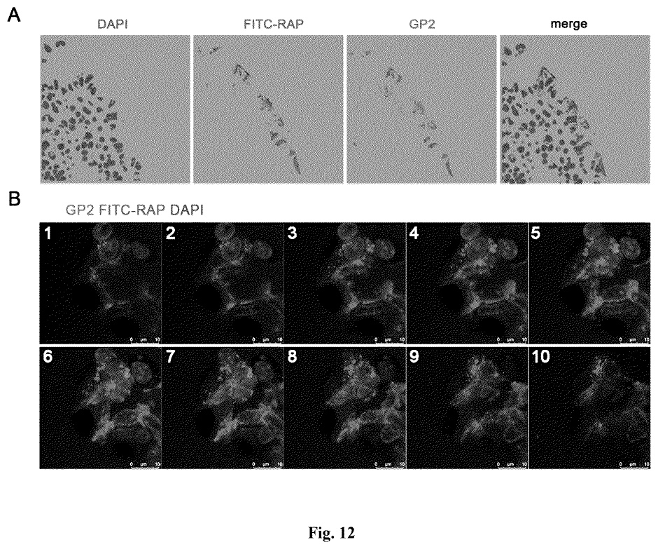

Transport of gut luminal molecules through M cells is thought to occur by a combination of non-specific transcytosis, receptor-mediated transcytosis, and extension of DC protrusions through M cell transcellular pores. Non-specific transcytosis is unlikely to make a major contribution to the system because the present inventors observed no transport of the control polysaccharide DOP. Confocal microscopic images of frozen sections of PPs isolated from mice 1 h after oral administration of FITC-RAP showed follicle dendritic cells (FDCs) receiving FITC-RAP after transcytosis through M cells, but not by capturing material through extending long processes (FIG. 11A-B). These observations implicated receptor-mediated transcytosis. Consistent with receptor-mediated uptake, the present inventors observed that FITC-RAP was precisely co-localized with GP2 receptors on the M cell apical surface in frozen sections (FIG. 12A). A series of confocal microscopic images of the whole-mount staining from apical to basal cells of the M-cell surface showed that FITC-RAP accumulating around GP2 was internalized into M cells (FIG. 12B). GP2 is a known receptor for targeted transcytosis by M-cells, so the present inventors hypothesized that the GP2 receptor of M cells might mediate the specific transcytosis of polysaccharides.

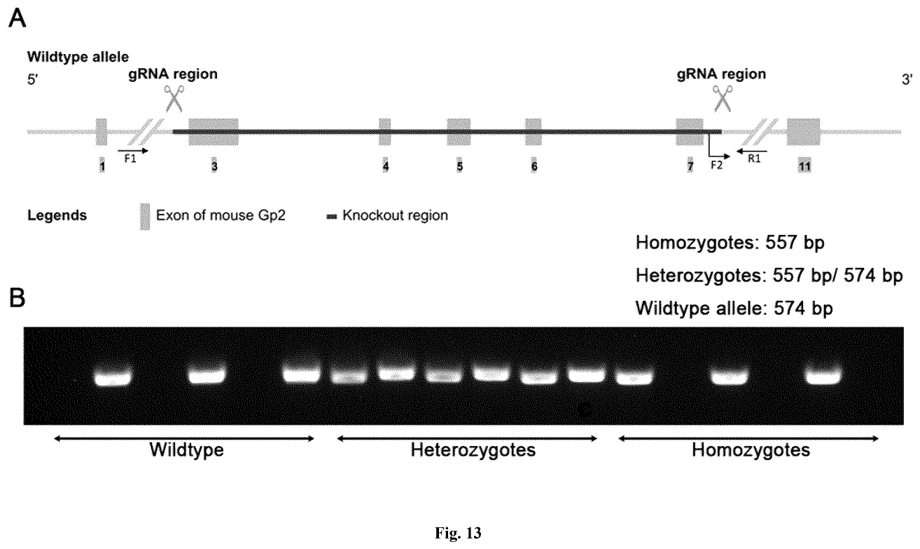

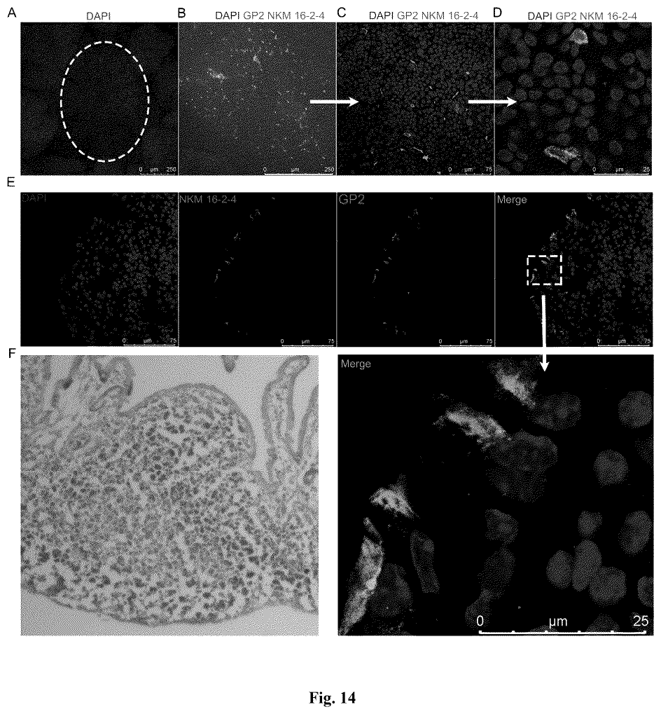

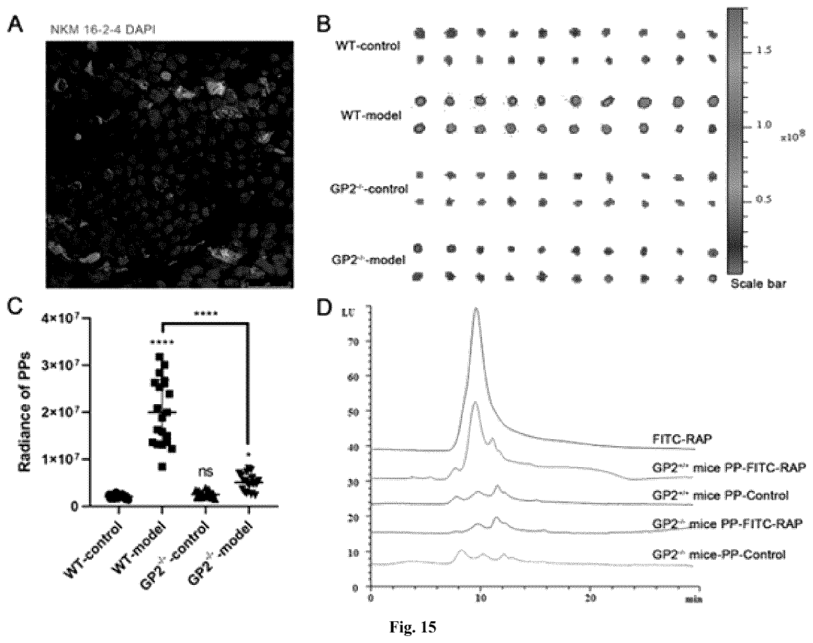

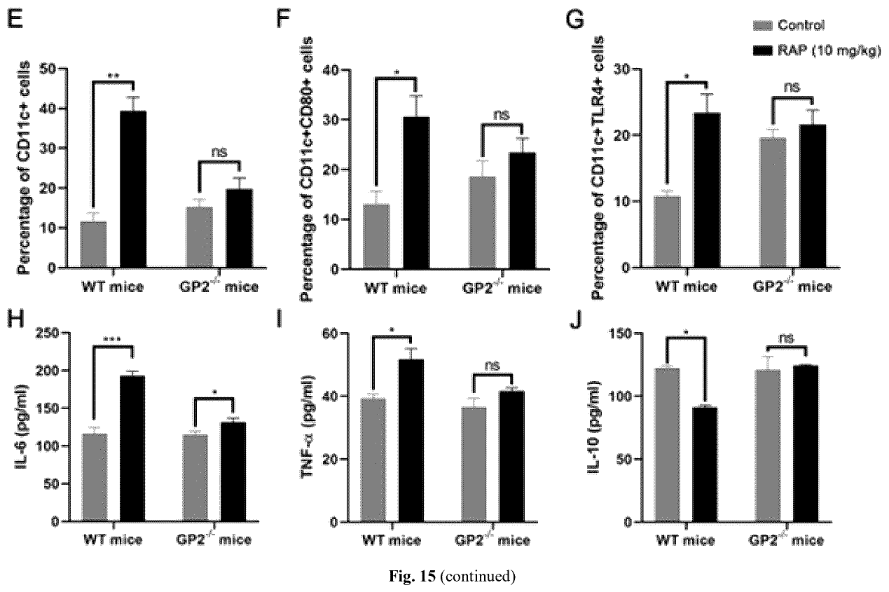

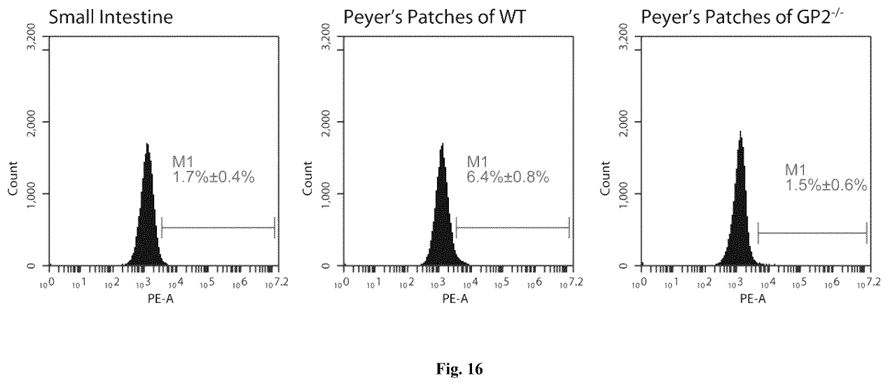

To test the hypothesis, the present inventors developed a GP2-deficient (GP2^{-/-}) mice model (FIG. 13A) and authenticated it by a PCR assay (FIG. 13B). The present inventors firstly confirmed that both GP2 and NKM16-2-4 could be used to mark M cells in wild-type (WT) mice (FIG. 14A-F). The whole-mount staining of PPs isolated from GP2^{-/-} mice showed that NKM 16-2-4 positive signal was still detected (FIG. 15A) while GP2 signal was not (FIG. 16). This indicates M cells still exist in the PPs of GP2^{-/-} mice. The present inventors then used the ligated loop assay to compare the accumulation of FITC-RAP between WT mice and GP2^{-/-} mice. IVIS Lumina XR Small Animal Images showed that the positive signals of FITC-RAP in PPs significantly decreased in the GP2^{-/-} mice (compares FIG. 15B and 15C). This decrease was further confirmed by HPGPC-FLD analysis of PP-associated fluorescence (FIG. 15D).

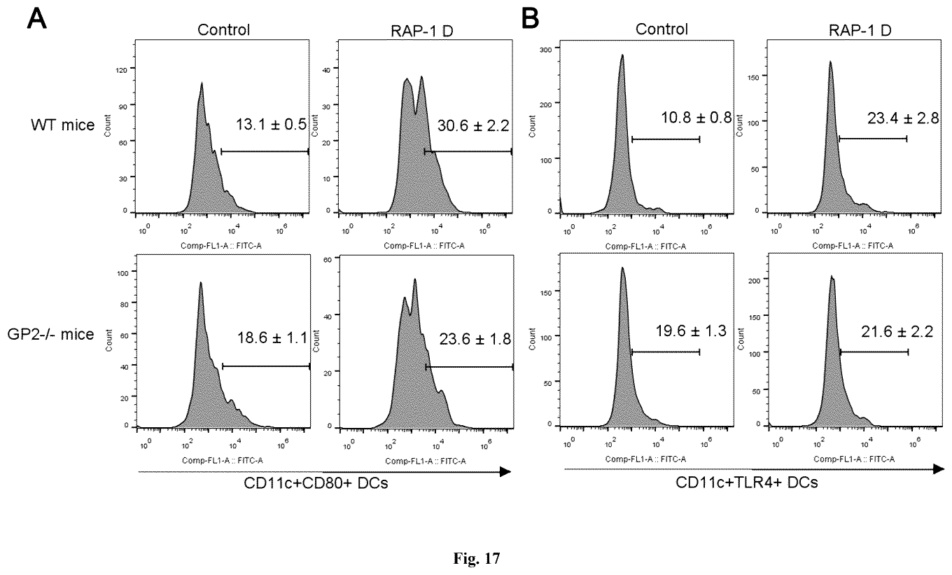

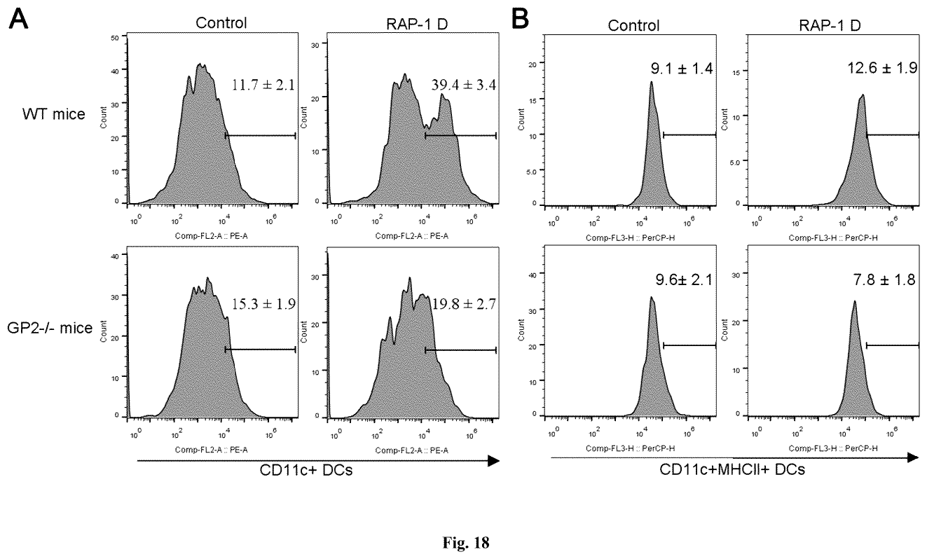

To determine if GP2 deficiency affects RAP's immunomodulatory activity, the present inventors compared signaling responses in immune cells between WT and GP2^{-/-} mice (FIG. 15E-J, FIG. 17A-B and FIG. 18A-B). Among the pathways measured, RAP induced strong signaling in WT mice but no statistically significant changes in GP2^{-/-} mice. These data strongly suggest that GP2-mediated M-cell transcytosis is necessary for RAP to exhibit the therapeutic activity.

### M Cell-Mediated Transcytosis of RAP in the Human Intestine

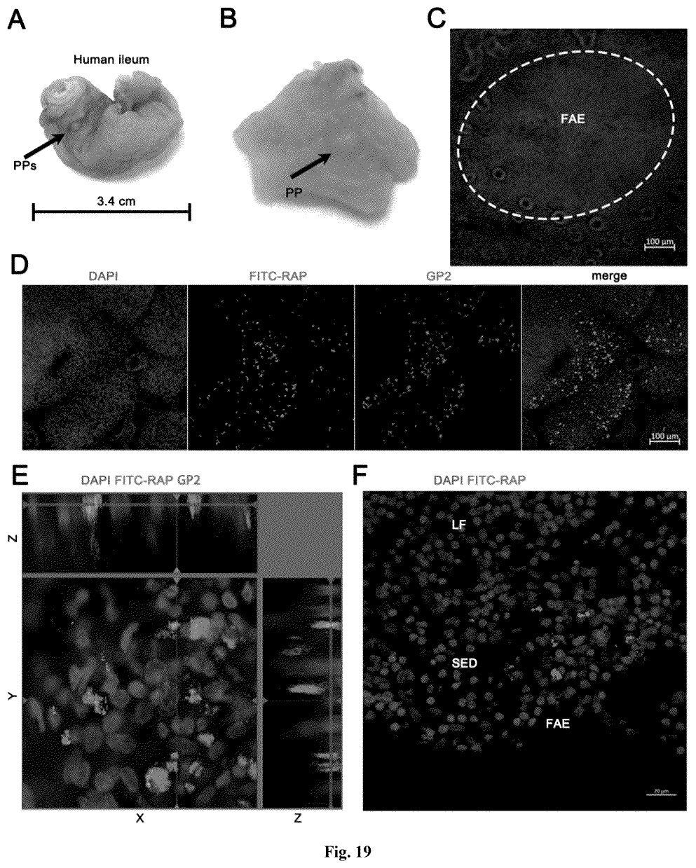

To investigate if the findings can extend to humans, the present inventors collected live distal ileum explants from surgery patients and screened them for PP domes (FIG. 19A and 19B). The present inventors incubated the PP-domes with FITC-RAP and then performed whole-mount staining. The dome zone of PPs is denoted by the white circle (FIG. 19C). GP2 was used as an M-cell marker. FITC-RAP was recruited of puncta, most of which were positive for GP2 (FIG. 19D). The two signals were not precisely coincident, which might be attributable to the dynamic transcytosis procedure. Three-dimensional images further confirmed and demonstrated the dynamic transport of FITC-RAP in M cells from the apical surface to the subepithelial dome of PPs (FIG. 19E). Confocal microscopic images of frozen sections of PPs confirmed that FITC-RAP could be transported into PPs (FIG. 19F). Taken together, these findings demonstrate that RAP can be transported by human M cells into PPs.

### DISCUSSION

The present inventors' data provide the first example of an orally dosed polysaccharide-based medicinal macromolecule undergoing delivery to the mucosal immune system by targeted transcytosis through M-cells, with important implications for the pharmacology of medicinal polysaccharides and oral delivery of future macromolecule drugs. Polysaccharides are among the most important active components of many TCMs, and it has long been mysterious how they can achieve systemic therapeutic effects despite extremely low oral bioavailability. The present invention shows that a representative medicinally active polysaccharide, RAP, accesses the immune system by receptor-mediated transcytosis through M-cells. Similar pathways may be used by other TCM macromolecules dosed orally.

This targeted delivery mode successfully bridges the gap between the in vitro and in vivo bioactivities of orally administrated polysaccharides. Many in vivo studies show oral polysaccharides' diverse immunity-targeting bioactivities such as antitumor and alleviating diabetes, and many in vitro studies address the underlying molecular mechanisms. However, few in vivo and in vitro studies could be linked together to make a compelling story because of the gut barrier. The present invention shows that intact polysaccharides could directly enter the host in the small intestine through M cells, supporting a possibility of direct contact between polysaccharides and cells in vivo. Indeed, the present inventors have previously observed that orally dosed RAP occurred in the bone marrow and connected with CD34+ stem cells, contributing to its protection from chemotherapy-induced myelosuppression. The M cell-mediated delivery thus reveals a targeted gateway for polysaccharides to meet immune cells directly in vivo, which provides scientific proof for the meaning of in vitro studies of polysaccharides.

Although gut microbiota plays an essential role in the bioactivities of many polysaccharides, here RAP has a gut microbiota-independent story. Unlike the initiation of gut microbiota-induced immune responses located in the large intestine, the present inventors' data showed that orally administrated RAP could directly trigger immune responses of PPs in several hours. The quick immunomodulatory effect is supportive of studies showing that M cell-mediated antigen sampling could complete about 15 minutes. Combining results that FMT did not affect the growth of the 4T1 tumor model, the present inventors speculate that RAP-induced immune responses in PPs are gut microbiota -independent, consistent with functions of polysaccharides on bile acid signaling and immune modulation. Although some polysaccharides were also found to enter PPs and induce immune responses directly, their entry was considered through M cell-mediated non-specific transcytosis. The present invention shows that the transcytosis of RAP is selective, which is GP2-dependent. The selective M cell-mediated delivery of RAP further thus suggests a novel immunomodulatory mechanism of polysaccharide in vivo, driven by a gut microbiota-independent way.

Perhaps an exciting implication of the present inventors' disclosure is for the development of future oral drug formulations. Oral drug delivery is the most preferred route of therapeutics administration. Despite these benefits, oral delivery of macromolecular therapeutics is limited by physicochemical conditions in the stomach, including acidic pH, gastric enzymes, mucus layer, and an epithelial cell layer, which are incompatible with the therapeutic's physical attributes molecules, e.g., surface charge and molecular size. Nanomaterials have been used to deliver macromolecules; however, they suffer from disadvantages such as the need to use organic solvents during synthesis, which may be denaturing the proteins, toxicity at large doses, limited loading capacity, and no specificity. The field of oral vaccination has long recognized the need for M-cell targeting functionality to direct vaccine molecules through M-cells to the mucosal immune system. Several targeting molecules have been explored in this context. So far, most have been proteins or peptides which are likely to suffer degradation during passage through the stomach and gut. RAP targets efficiently to M-cells in the present invention and are known to be non-toxic, indeed beneficial, to gut health. The present inventors propose that intact RAP, or chemically defined fragments, can serve as an efficient, safe, and chemically stable targeting agent to deliver macromolecules drugs and vaccines via oral dosing.

### CONCLUSION

The present invention provides a delivery target site of polysaccharides in the small intestine: receptor-dependent transcytosis of polysaccharides by M cells into Peyer's patches. These results elucidate a long-standing mystery of how polysaccharides are delivered after oral administration and highlight a blood-independent and gut microbiota-independent approach for indigestible macromolecules to perform in vivo. The present invention also provides a receptor-dependent strategy for delivering future macromolecular drugs and vaccines targeting the mucosal immune system.

### INDUSTRIAL APPLICATION

The present invention provides a traditional Chinese Medicine derived macromolecule as a delivery agent for triggering antitumor immunity. In particular, the present invention provides a targeting agent, polysaccharides of Radix Astragali, to deliver macromolecules drugs and vaccines via oral dosing.

1. A method of delivering a drug into a lymphatic system in a subject in need thereof, the method comprising administering a Radix Astragali polysaccharide and the drug to the subject, wherein the drug is transported via a lymphatic system at small intestine mucosa or lung mucosa of the subject by M-cell GP2-mediated transcytosis.

2. The method of claim 1, wherein the Radix Astragali polysaccharide is a branched polysaccharide with an average molecular weight of 100-1500 kDa comprising Rha, Ara, Glc, Gal and GalA in a molar ratio of 0.01-0.03:1.00:0.20-10.00:0.01-0.50:0.01-0.30.

3. The method of claim 2, wherein the branched polysaccharide comprises a backbone and one or more sidechains, wherein the backbone comprises 1,2,4-linked Rhap, α-1,4-linked Glcp, α-1,4-linked GalAp6Me, or β-1,3,6-linked Galp; and the one more sidechains comprise α-T-Araf, α-1,5-linked Araf with O-3, T-linked Araf, T-linked Glcp, and T-linked Galp.

4. The method of claim 1, wherein the Radix Astragali polysaccharide is covalently bonded to the drug via an optional linker to form a covalent conjugate of the Radix Astragali polysaccharide and the drug.

5. The method of claim 1, wherein the drug comprises a small molecule, a peptide, an antibody, an antibody fragment, or a vaccine.

6. The method of claim 1, wherein the subject is a human or a non-human mammal.

7. The method of claim 1, wherein the Radix Astragali polysaccharide is formulated into an oral administrable composition or an aerosolized composition.

8. The method [[use]] of claim 4, wherein the covalent conjugate of the Radix Astragali polysaccharide and the drug is formulated into an orally administrable composition or an aerosolized composition.

9. The method of claim 7, wherein the orally administrable composition is formulated into a solid, a powder, a liquid, a gel, a capsule, a tablet, a pill, a pellet, or a particle.

10. The method of claim 7, wherein the aerosolized composition is formulated into particles or aerosols.

11. (canceled)

12. A delivery agent comprising a polysaccharide specifically targeting a receptor of microfold (M) cells, which when the polysaccharide is covalently bonded to a drug via an optional linker to form a polysaccharide drug conjugate, the resulting polysaccharide drug conjugate is capable of being transported via the lymphatic system of a subject via receptor-mediated transcytosis.

13. The delivery agent of claim 12, wherein the polysaccharide comprises one or more Radix Astragali polysaccharides, wherein the one or more Radix Astragali polysaccharides are branched polysaccharides with an average molecular weight of 100-1500 kDa comprising Rha, Ara, Glc, Gal and GalA in a molar ratio of 0.01-0.03:1.00:0.20-10.00:0.01-0.50:0.01-0.30.

14. The delivery agent of claim 13, wherein each of the branched polysaccharides comprises a backbone and one or more sidechains, wherein the backbone comprises 1,2,4-linked Rhap, α-1,4-linked Glcp, α-1,4-linked GalAp6Me, or β-1,3,6-linked Galp; and the one more sidechains comprise α-T-Araf, α-1,5-linked Araf with O-3, T-linked Araf, T-linked Glcp, and T-linked Galp.

15. (canceled)

16. (canceled)

17. (canceled)

18. (canceled)

19. (canceled)

20. A pharmaceutical composition comprising the delivery agent according to claim 13 and at least one pharmaceutically acceptable excipient or pharmaceutically acceptable carrier.

21. (canceled)

22. (canceled)

23. A method comprising: administering the delivery agent according to claim 13 to a subject in need thereof.

24. (canceled)

25. (canceled)

26. The method of claim 23, wherein the pharmaceutical composition also comprises a drug other than the delivery agent.

27. The method of claim 23, wherein the pharmaceutical composition is formulated into an orally administrable form.

28. (canceled)

29. The method of claim 23, wherein the pharmaceutical composition is formulated into an aerosolized form.

30. (canceled)

31. The method of claim 23, wherein the drug comprises a small molecule, a peptide, an antibody, an antibody fragment, or a vaccine.

32. (canceled)
